(19) United States
(12) Patent Application Publication
(10) Pub. No.: US 2024/0050249 A1
(43) Pub. Date: Feb. 15, 2024

(54) DELIVERY APPARATUS AND CONTROL DEVICE

(71) Applicants: Deda LOU, Shenzhen, Guangdong (CN); SHENZHEN CHUANGXIN MEDICAL TECHNOLOGY CO., LTD., Shenzhen, Guangdong (CN)

(72) Inventors: Deda LOU, Shenzhen, Guangdong (CN); Yucheng PENG, Shenzhen, Guangdong (CN); Chengming SU, Shenzhen, Guangdong (CN)

(73) Assignees: Deda LOU, Shenzhen, Guangdong (CN); SHENZHEN CHUANGXIN MEDICAL TECHNOLOGY CO., LTD., Shenzhen, Guangdong (CN)

(21) Appl. No.: 17/632,197

(22) PCT Filed: Oct. 30, 2020
(86) PCT No.: PCT/CN2020/125483

§ 371 Date: Feb. 1, 2022

(30) Foreign Application Priority Data
Jul. 10, 2020 (CN) 202010662210.2

(51) Int. Cl. A61F 2/966

(52) U.S. Cl. CPC A61F 2/966

(57) ABSTRACT

The present application disclosed a delivery apparatus and a control device. The control device includes a fixing unit and a movable unit, the fixing unit includes a screw rod provided with an accommodating hole and a sliding groove communicating with the accommodating hole; the movable unit includes a connecting member, a first handle, a mating member, and a control member. The control device can control the fast movement and the slow movement of the connecting member, thereby facilitating the realization of the speed control of the release of the vascular stent. The delivery apparatus uses the control device.

### TECHNICAL FIELD

The present application relates to the technical field of medical equipment, and more particularly to a delivery apparatus and a control device.

### BACKGROUND

In recent years, some scholars have begun to try to use endovascular exclusion to treat aortic diseases, and related chimney and fenestration techniques have begun to achieve some results, and the results are encouraging. However, due to the particularity of the anatomical structure of the arch of the aorta and the complexity of hemodynamics, the implantation of the vascular stent into the body requires the use of a delivery apparatus for delivery.

Traditional delivery apparatuses generally adopt a hollow inner tube and an outer tube that are sleeved with each other. The hollow portion of the inner tube is convenient for the guide wire to pass through the tube during the operation, and the vascular stent is arranged between the inner tube and the outer tube. Due to the structural defects of the traditional delivery apparatuses, the release process of the vascular stent is too slow, and the operation time is too long, which is not conducive to postoperative recovery, and may even cause postoperative complications.

### SUMMARY

Based on this, it is necessary to provide a delivery apparatus and a control device. The control device can control the fast movement and the slow movement of the connecting member, which is beneficial to realize the speed control of the release of the vascular stent. The delivery apparatus adopts the control device, which can realize the speed control of the vascular stent, which is beneficial to shorten the operation time.

The technical solution is as follows:

In a first aspect, the present application provides a control device, which includes a fixing unit and a movable unit; the fixing unit includes a screw rod provided with an accommodating hole and a sliding groove communicating with the accommodating hole, a depth of the accommodating hole and a length of the sliding groove are arranged along an axis direction of the screw rod; and the movable unit includes a connecting member, a first handle, a mating member, and a control member; the connecting member is provided with a first connecting body and a second connecting body fixed to the first connecting body, the first connecting body is arranged in the accommodating hole, and the first connecting body is slidingly engaged with the screw rod, the second connecting body is rotatably connected to the first handle passing through the sliding groove, the first handle is hollow and is sleeved on an outside of the screw rod, and movable relative to the screw rod, the mating member is arranged in the first handle, and the mating member is provided with an internal thread structure engaged with the screw rod, the control member is movably arranged on the first handle, and the first handle drives the mating member to rotate through the control member; when the control member is in a first position, the internal thread structure is separated from the screw rod; and when the control member is in a second position, the internal thread structure is engaged with the screw rod through a thread transmission.

When the above-mentioned control device is used, the connecting member, the mating member and the control member are integrated on the first handle, and the first connecting body of the connecting member is arranged in the accommodating hole and is directly or indirectly slidingly engaged with the screw rod. At the same time, the first handle is rotatably connected to the connecting member through the second connecting body, such that the first handle can drive the connecting member to move in the axial direction of the screw rod as well as rotate relative to the connecting member. The first connecting body is connected with the delivery tube. Specifically, when applied to a delivery apparatus, when the vascular stent needs to be released quickly, the control member moves to the first position, at this time, the internal thread structure is separated from the screw rod, so that the mating member can move relative to the axial direction of the screw rod, that is, the moving of the first handle can drive the control member, the mating member and the connecting member to move in the axial direction of the screw rod. The connecting member is used to quickly pull the delivery tube so that the vascular stent is released quickly; when the vascular stent is released to a certain extent, fine release control is required, at this time, the control member can be moved to the second position, and the internal thread structure is engaged with the screw rod through a thread transmission, so that the mating member and the screw rod are screwed together. At this time, rotating the first handle can drive the mating member to move along the axis direction of the screw rod, that is, driving the first handle, the control member and the connecting member to move along the axial direction of the screw rod, and the moving distance of the connecting member is controlled by rotating the first handle to realize the fine release of the vascular stent. In this way, the control device can control the fast movement and the slow movement of the connecting member, thereby facilitating the realization of the speed control of the release of the vascular stent.

The further describing of the technical solution are following: In one of embodiments, the movable unit further comprises a sleeve, the sleeve is sleeved on the outside of the screw rod, and the sleeve is provided with a through hole, the mating member is rotatably arranged on the sleeve, and the internal thread structure is capable of passing through the through hole to be engaged with the screw rod, the mating member is further provided with a protrusion spaced apart from the internal thread structure in a circumferential direction, the protrusion is arranged in a direction away from the screw rod, and the control member is provided with a pressing body and an extruding body spaced apart from the pressing body;
when the control member is in the first position, the extruding body is offset from the mating member, and the pressing body is press-fitted with the protrusion, such that the internal thread structure is separated from the screw rod; and
when the control member is in the second position, the pressing body is offset from the protrusion, and the extruding body is press-fitted with the mating member, such that the internal thread structure is engaged with the screw rod through a thread transmission, and the first handle is capable of driving the control member and the mating member to rotate.

In one of embodiments, the control member is arranged in the sleeve and capable of being elastically reset, and the control member is capable of being automatically reset to the second position under an elastic force; and an inclined surface introduction structure is provided between the extruding body and the mating body.

In one of embodiments, the mating member is arranged on the screw rod and capable of being elastically reset, and the control member is provided with an insertion portion;
when the control member is in the first position, the insertion portion is inserted between the internal thread structure and the screw rod, such that the internal thread structure is separated from the screw rod; and
when the control member is in the second position, the insertion portion is offset from both the internal thread structure and the screw rod, and the mating member is reset under an elastic force, such that the internal thread structure is engaged with the screw rod through a thread transmission.

In one of embodiments, the fixing unit further comprises a second handle, the second handle is fixed on the screw rod, and the first handle is movable relative to the second handle.

In one of embodiments, the control device further includes a bending unit, the bending unit includes a driving member and a flexible traction member, the driving member is movably disposed on the first handle, and the driving member is movable with the first handle, and one end of the flexible traction member is fixed on the driving member.

In one of embodiments, the driving member is rotatable relative to the first handle, and the driving member is provided with a connecting sleeve sleeved on the screw rod and a force applying portion fixed on an outside of the connecting sleeve; one end of the flexible traction member is fixed on the connecting sleeve; or the connecting sleeve is provided with an external thread structure, and the bending unit further includes a third handle fixedly connected with the first handle and a threaded sleeve that is screwed and engaged with the external thread structure, the third handle is hollow, the threaded sleeve is arranged in the third handle and is slidably connected to the third handle, one end of the flexible traction member is fixed on the threaded sleeve.

In one of embodiments, the fixing unit further includes a guide rod, the guide rod is fixed in the screw rod, and the first connecting body is provided with a mating hole that is slidingly fitted with the guide rod.

In one of embodiments, the guide rod is provided with a flow channel, and the fixing unit further includes a guide tube in communicated with the flow channel, and one end of the guide tube is fixed to the screw rod and is communicated with outside.

In another aspect, the present application further provides a delivery apparatus, which includes the control device above-mentioned, the first connecting body is provided with a connection portion, and the delivery apparatus further includes a delivery tube for delivering a vascular stent, and one end of the delivery tube is fixedly connected with a connecting member through the connection portion.

When the delivery apparatus is in used, when the vascular stent needs to be released quickly, the control member moves to the first position, at this time, the internal thread structure is separated from the screw rod, so that the mating member can move relative to the axial direction of the screw rod, that is, the moving of the first handle can drive the control member, the mating member and the connecting member to move in the axial direction of the screw rod. The connecting member is used to quickly pull the delivery tube so that the vascular stent is released quickly; when the vascular stent is released to a certain extent, fine release control is required, at this time, the control member can be moved to the second position, and the internal thread structure is engaged with the screw rod through a thread transmission, so that the mating member and the screw rod are screwed together. At this time, rotating the first handle can drive the mating member to move along the axis direction of the screw rod, that is, driving the first handle, the control member and the connecting member to move along the axial direction of the screw rod, and the moving distance of the connecting member is controlled by rotating the first handle to realize the fine release of the vascular stent. The delivery apparatus uses the control device, and the speed control of the release of the vascular stent can be realized, which is beneficial to shorten the operation time.

### BRIEF DESCRIPTION OF THE DRAWINGS

FIG. 1 is a schematic view of an embodiment of a delivery apparatus;

FIG. 2 is a use schematic view of the delivery apparatus shown in FIG. 1;

FIG. 3 is a structural explosion view of the delivery apparatus shown in FIG. 1;

FIG. 4 is a partial cross-sectional schematic view of a control device shown in FIG. 1;

FIG. 5 is a cross-sectional schematic view of the control device shown in FIG. 1 from another sight angle;

FIG. 6 is a structural schematic view of the control device shown in FIG. 4 with a part of the first handle is hidden;

FIG. 7 is a structural schematic view of the control device shown in FIG. 6 from another sight angle;

FIG. 8 is a partial cross-sectional schematic view of a bending unit shown in FIG. 1; and

FIG. 9 is a partial enlarged view of the control device shown in FIG. 3.

The reference signs in the drawings are listed:

100—fixing unit; 110—screw rod; 112—accommodating hole; 114—sliding groove; 120—second handle; 130—guide rod; 140—guide tube; 200—movable unit; 210—first handle; 220—connecting member; 222—first connecting body; 202—connection portion; 204—mating hole; 224—second connecting body; 230—mating member; 232—internal thread structure; 234—protrusion; 236—inclined surface introduction structure; 240—control member; 242—pressing body; 244—extruding body; 250—sleeve; 252—through hole; 300—bending unit; 310—driving member; 312—connecting sleeve; 302—external thread structure; 314—force application portion; 320—flexible traction member; 330—third handle; 340—threaded sleeve; 400—delivery tube.

The accompanying drawings constituting a part of the present application are used to provide a further understanding of the present application, the exemplary embodiments and descriptions of the present application are used to explain the present application, and do not constitute an improper limitation of the present application.

In order to explain the technical solutions in the embodiments of the present application more clearly, the following will briefly introduce the accompanying drawings used in the description of the embodiments. Obviously, the accompanying drawings in the following description are only some embodiments of the present application. For those skilled in the art, other drawings can be obtained from these drawings without creative work.

### DETAILED DESCRIPTION

In order to make the objectives, technical solutions, and advantages of the present application clearer, the present application will be further described in detail below with reference to the accompanying drawings and specific embodiments. It should be understood that the specific embodiments described herein are only used to explain the present application, and do not limit the protection scope of the present application.

Unless otherwise defined, all technical and scientific terms used herein have the same meaning as commonly understood by those skilled in the technical field of the present application. The terminology used in the specification of the present application herein is only for the purpose of describing specific embodiments, and is not intended to limit the present application. The term “and/or” as used herein includes any and all combinations of one or more related listed items.

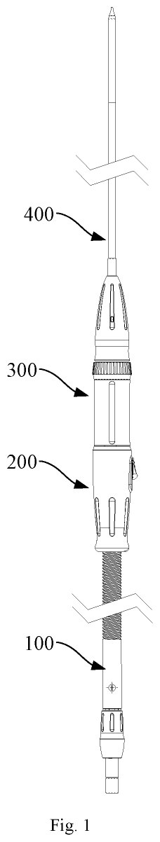

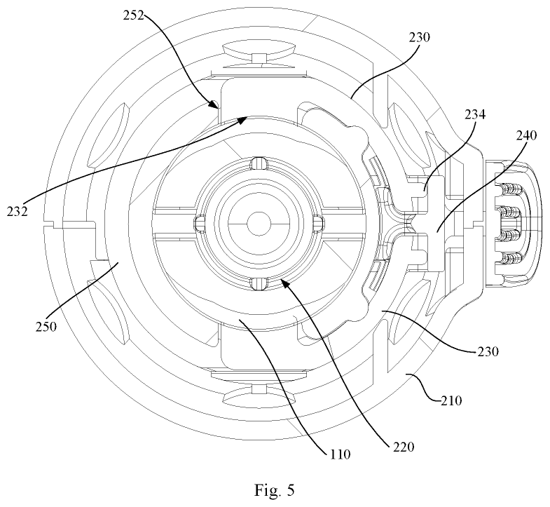

As shown in FIGS. 1 and 4 to 7, a control device is provided, which includes a fixing unit 100 and a movable unit 200.

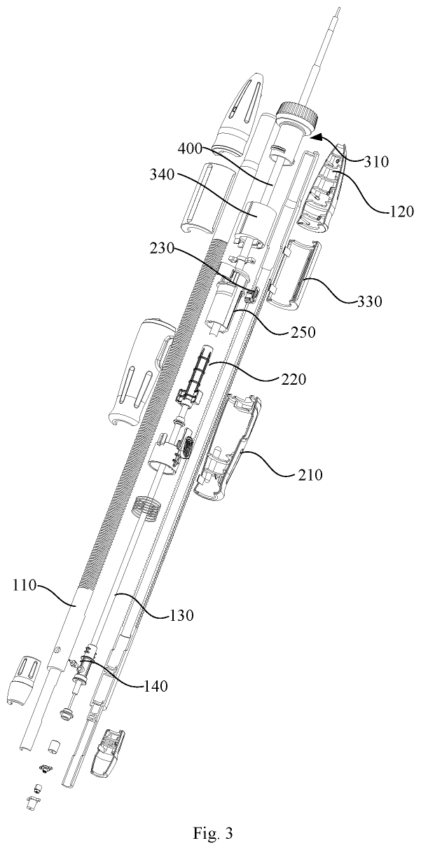

As shown in FIGS. 3, 4 and 7, the fixing unit 100 includes a screw rod 100. The screw rod 100 is provided with an accommodating hole 112 and a sliding groove 114 communicating with the accommodating hole 112. The depth of the accommodating hole 112 and the length of the sliding groove 114 are arranged along the axial direction of the screw rod 100.

As shown in FIGS. 3 to 7, the movable unit 200 includes a connecting member 220, a first handle 210, a mating member 230, and a control member 240. The connecting member 220 is provided with a first connecting body 222 and a second connecting body 224 fixed to the first connecting body 222. The first connecting body 222 is arranged in the accommodating hole 112, the first connecting body 222 is slidingly engaged with the screw rod 100, the second connecting body 224 passing through the sliding groove 114 and is rotatably connected with the first handle 210, the first handle 210 is hollow and is sleeved in the outer side of the screw rod 100 and can move relative to the screw rod 100. The mating member 230 is arranged in the first handle 210, the mating member 230 is provided with an internal thread structure 232 that fits the screw rod 100, and the control member 240 is movably arranged at the first handle 210. Among them, when the control member 240 is in the first position, the internal thread structure 232 is separated from the screw rod 100; when the control member 240 is in the second position, the internal thread structure 232 is engaged with the screw rod 100 through a thread transmission, and the first handle 210 can drive the control member 240 and the mating member 230 to rotate.

When the above-mentioned control device is used, the connecting member 220, the mating member 230 and the control member 240 are integrated on the first handle 210, and the first connecting body 222 of the connecting member 220 is arranged in the accommodating hole 112 and is directly or indirectly slidingly engaged with the screw rod 110. At the same time, the first handle 210 is rotatably connected to the connecting member 220 through the second connecting body 224, such that the first handle 210 can drive the connecting member 220 to move in the axial direction of the screw rod 110 as well as rotate relative to the connecting member 220. The first connecting body 222 is connected with the delivery tube 400. Specifically, when applied to a delivery apparatus, when the vascular stent needs to be released quickly, the control member 240 moves to the first position, at this time, the internal thread structure 232 is separated from the screw rod 110, so that the mating member 230 can move relative to the axial direction of the screw rod 110, that is, the moving of the first handle 210 can drive the control member 240, the mating member 230 and the connecting member 220 to move in the axial direction of the screw rod 110. The connecting member 220 is used to quickly pull the delivery tube 400 so that the vascular stent is released quickly; when the vascular stent is released to a certain extent, fine release control is required, at this time, the control member 240 can be moved to the second position, and the internal thread structure 232 is engaged with the screw rod 110 through a thread transmission, so that the mating member 230 and the screw rod 110 are screwed together. At this time, rotating the first handle 210 can drive the mating member 230 to move along the axis direction of the screw rod 110, that is, driving the first handle 210, the control member 240 and the connecting member 220 to move along the axial direction of the screw rod 110, and the moving distance of the connecting member 220 is controlled by rotating the first handle 210 to realize the fine release of the vascular stent. In this way, the control device can control the fast movement and the slow movement of the connecting member 220, thereby facilitating the realization of the speed control of the release of the vascular stent.

It should be noted that there can be multiple forms of fitting between the “control member 240” and the “mating member 230”, as long as the separation and the transmission between the internal thread structure 232 and the screw rod 110 can be c switched.

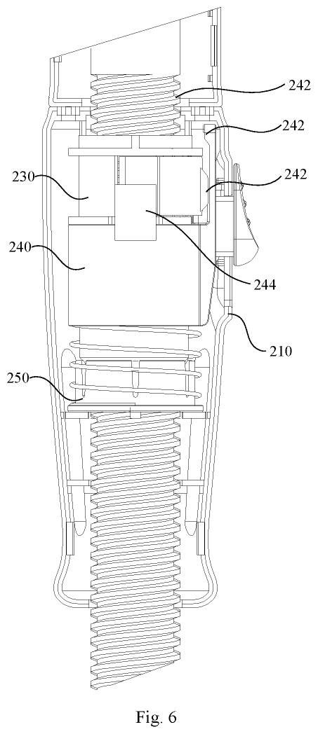

Specifically in this embodiment, as shown in FIGS. 6, 7 and 9, the movable unit 200 further includes a sleeve 250, which is sleeved on the outside of the screw rod 100, the sleeve 250 is provided with a through hole 252, and the mating member 230 can be rotatably arranged on the sleeve 250, and the internal thread structure 232 can pass through the through hole 252 to fit with the screw rod 100. The mating member 230 is further provided with a protrusion 234 spaced from the internal thread structure 232 in the circumferential direction. The protrusion 234 is arranged in a direction f away from the screw rod 110, the control member 240 is provided with a pressing body and an extruding body arranged apart from the pressing body. When the control member 240 is in the first position, the extruding body is offset from the mating member 230, and the pressing body is press-fitted with the protrusion 234, so that the internal thread structure 232 is separated from the screw rod 100; when the control member 240 is in the second position, the pressing body is offset from the extension 234, the extruding body is press-fitted with the mating body, so that the internal thread structure 232 is engaged with the screw rod 100 through a thread transmission.

In this way, the mating member 230 can be rotatably arranged on the sleeve 250 to form a seesaw structure. When the control member 240 moves to the first position, the extruding body is offset from the mating member 230, and the pressing body is press-fitted with the protrusion 234, so that the internal thread structure 232 is raised and separated from the screw rod 100; when the control member 240 moves to the second position, the pressing body is offset from the protrusion 234, the extruding body is press-fitted with the mating body, and the internal thread structure 232 is reset, so that the internal thread structure 232 is engaged with the screw rod 100 through a thread transmission. In this process, only by moving the control member 240 along the axial direction of the screw rod 100 can switch between the first position and the second position, and switch the separation and transmission cooperation between the internal thread structure 232 and the screw rod 100.

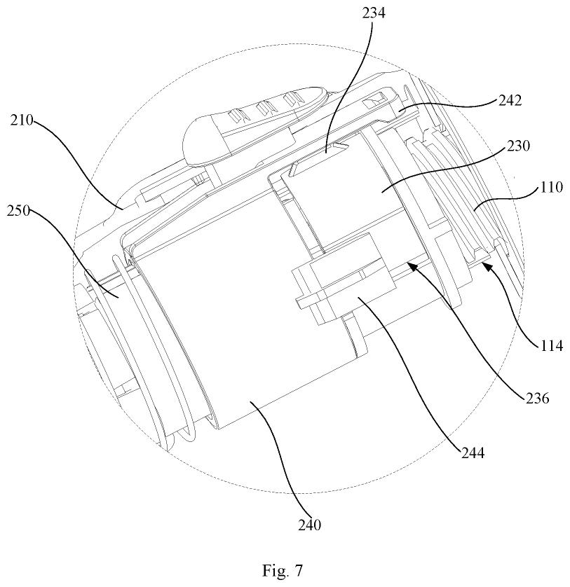

Based on the above embodiment, as shown in FIGS. 6 and 7, in one embodiment, the control member 240 can be elastically reset on the sleeve 250, and under the elastic force, the control member 240 can be automatically reset to the second position. There is an inclined surface introduction structure 236 is provided between the extruding body and the mating member. In this way, under the elastic force, the control member 240 can automatically reset to the second position, so that the mating member 230 and the screw rod 100 are screwed together, and then the first handle 210 can be rotated to drive the connecting member 220 slowly along the axial direction of the screw rod 100, the movement facilitates the fine control of the release of the vascular stent. At the same time, by arranging the inclined surface introduction structure 236, it is convenient to press-fit the extruding body to the mating body, so that the internal thread structure 232 is engaged with the screw rod 100 through a thread transmission. The inclined surface introduction structure 236 can be arranged on the extruding body or/and the mating body to play the role of first introduction and gradual extrusion.

Of course, in another embodiment, the control member 240 includes a first pressing portion (not shown) and a second pressing portion (not shown); when the first pressing portion is pressed down to the first position, the second pressing portion is not at the second position, the pressing body is press-fitted with the protrusion 234, so that the internal thread structure 232 is raised and separated from the screw rod 100; when the second pressing portion is pressed down to the second position, the first pressing portion is not at the first position, the pressing body is offset from the protrusion 234, and the extruding body is press-fitted the internal thread structure 232, so that the internal thread structure 232 is reset, and the internal thread structure 232 and the screw rod 100 are screwed together. In this way, the control member 240 can also be used to switch between the two positions by using the pressing principle.

The first pressing portion and the second pressing portion can be realized by using existing technologies such as indexing pins.

In further embodiment, the mating member 230 can be elastically reset and arranged on the screw rod 100, and the control member 240 is provided with an insertion portion (not shown). When the control member 240 is in the first position, the insertion portion is inserted between the internal thread structure 232 and the screw rod 100, so that the internal thread structure 232 is separated from the screw rod 100; when the control member is in the second position, the insertion portion is offset from both the internal thread structure 232 and the screw rod 100, and the mating member 230 is reset under the elastic force, so that the internal thread structure 232 is engaged with the screw rod 100 through a thread transmission. In this way, by automatically resetting the insertion portion and the mating member 230, it is also possible to switch the separation and the transmission cooperation between the internal thread structure 232 and the screw rod 100.

The insertion portion can be movably inserted between the internal thread structure 232 and the screw rod 100, or can be inserted between the internal thread structure 232 and the screw rod 100 by pressing, which can be selected according to actual needs.

On the basis of any of the foregoing embodiments, as shown in FIG. 3, in one embodiment, the fixing unit 100 further includes a second handle 120, the second handle 120 is fixed on the screw rod 100, and the first handle 210 can move relative to the second handle 120. In this way, by arranging the second handle 120, it is convenient for the operator to grab the second handle 120 to perform related operations on the first handle 210, and realize the movement and rotation of the first handle 210.

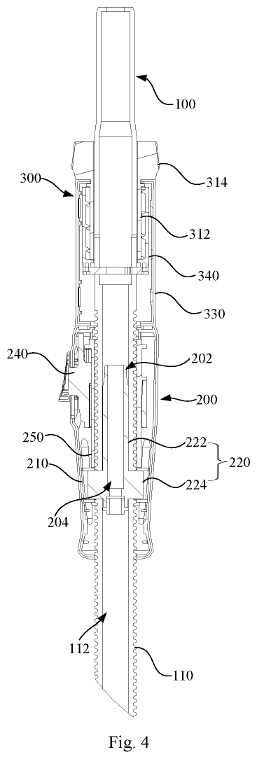

Based on any of the foregoing embodiments, as shown in FIGS. 4, 8 and 9, in one embodiment, the control device further includes a bending unit 300, and the bending unit 300 includes a driving member 310 and a flexible traction member 320, and the driving member 310 is movably arranged on the first handle 210, and the driving member 310 can move with the first handle 210, and one end of the flexible traction member 320 is fixed on the driving member 310. In this way, the other end of the flexible traction member 320 is fixedly connected to the free end of the delivery tube 400. When the delivery tube 400 enters the arch portion of the aorta, such that the driving member 310 is moved driving the flexible traction member 320 to pull the delivery tube 400 to bend, so that the delivery tube 400 enables the vascular stent to pass through the arch portion of the larger aorta smoothly and safely, overcomes the problem of easy damage to the arch portion of the aortic during the delivery of the vascular stent, and reduces the risk of surgery.

Further, in one embodiment, the driving member 310 can rotate relative to the first handle 210, and the driving member 310 is provided with a connecting sleeve 312 sleeved on the screw rod 100 and a force applying portion 314 fixed on the outside of the connecting sleeve 312, one end of the flexible traction member 320 is fixed on the connecting sleeve 312. In this way, when it is necessary to perform bending control, only need to rotate the force application portion 314 making the connecting sleeve 312 to rotate, which in turn drives the flexible traction member 320 to shrink and wrap around the connecting sleeve 312, so that the delivery tube 400 bends so as to smoothly pass through the curved portion of the blood vessel.

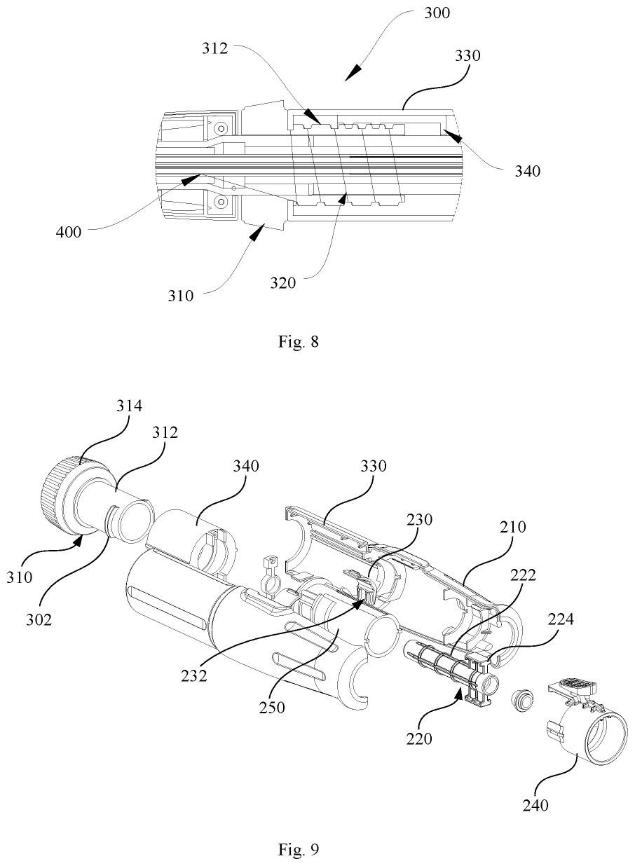

Alternatively, in another embodiment, as shown in FIGS. 8 and 9, the driving member 310 can rotate relative to the first handle 210, and the driving member 310 is provided with a connecting sleeve 312 sleeved on the screw rod 100 and a force application portion 314 fixed on the outer side of the connecting sleeve 312, the connecting sleeve 312 is provided with an external thread structure 302, and the bending unit 300 further includes a third handle 330 fixedly connected to the first handle 210, and a threaded sleeve 340 that is screwed to the external thread structure 302. The third handle 330 is hollow, and the threaded sleeve 340 is arranged in the third handle 330 and is slidably connected to the third handle 330, one end of the flexible traction member 320 is fixed on the threaded sleeve 340. In this way, when the bending control is required, only the force application portion 314 needs to be rotated making the connecting sleeve 312 to rotate, which in turn drives the threaded sleeve 340 to move along the axis of the connecting sleeve 312, and drives the flexible traction member 320 to be tightened, such that the delivery tube 400 is bended, so as to smoothly pass through the curved portion of the blood vessel.

It should be noted that the flexible traction member 320 is a traction rope or a traction chain.

Specifically, in the embodiment, the flexible traction member 320 is a traction rope, which is beneficial to save space, so that the volume of the delivery device can be smaller, especially the delivery tube 400 has a smaller volume, which is suitable for minimally invasive surgery.

On the basis of any of the above embodiments, as shown in FIG. 3, in one embodiment, the fixing unit 100 further includes a guide rod 130, the guide rod 130 is fixed in the screw rod 100, and the first connecting body 222 is provided with a mating hole 204 for sliding fit with the guide rod 130. In this way, through the sliding fit of the guide rod 130 and the mating hole 204, the connecting member 220 can slide relative to the screw rod 100, so that the connecting member 220 moves more smoothly.

Further, in one embodiment, the guide rod 130 is provided with a flow channel (not shown), and the fixing unit 100 further includes a guide tube 140 that communicates with the flow channel One end of the guide tube 140 is fixed on the screw rod 100 and communicated to the outside. In this way, the required reagent can be injected into the flow channel through the guide tube 140, and the reagent flows into the delivery tube 400 through the guide tube 140, the flow channel, and the mating hole 204, and flows out of the vascular stent, so that treatment can be better.

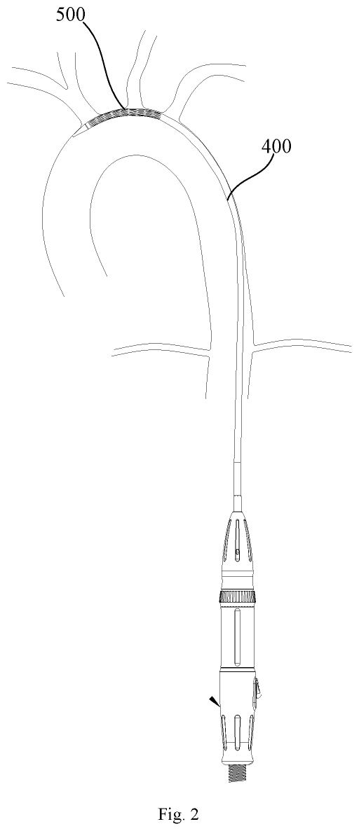

As shown in FIGS. 1 to 3, in one embodiment, a delivery apparatus is further provided, which includes the control device in any of the above embodiments, the first connecting body 222 is provided with a connection portion 202, and further includes a delivery tube 400, and one end of the delivery tube 400 is fixedly connected to the connecting member 220 through the connection portion 202.

When the delivery apparatus is in used, when the vascular stent needs to be released quickly, the control member 240 moves to the first position, at this time, the internal thread structure 232 is separated from the screw rod 110, so that the mating member 230 can move relative to the axial direction of the screw rod 110, that is, the moving of the first handle 210 can drive the control member 240, the mating member 230 and the connecting member 220 to move in the axial direction of the screw rod 110. The connecting member 220 is used to quickly pull the delivery tube 400 so that the vascular stent is released quickly; when the vascular stent is released to a certain extent, fine release control is required, at this time, the control member 240 can be moved to the second position, and the internal thread structure 232 is engaged with the screw rod 110 through a thread transmission, so that the mating member 230 and the screw rod 110 are screwed together. At this time, rotating the first handle 210 can drive the mating member 230 to move along the axis direction of the screw rod 110, that is, driving the first handle 210, the control member 240 and the connecting member 220 to move along the axial direction of the screw rod 110, and the moving distance of the connecting member 220 is controlled by rotating the first handle 210 to realize the fine release of the vascular stent. The delivery apparatus adopts the control device, which can realize the speed control of the vascular stent, which is beneficial to shorten the operation time.

Specifically, the delivery apparatus is fitted with a vascular stent and can be applied to the treatment of Stanford Type A aortic dissection. Stanford Type A acute aortic dissection type A (referred to as AADA) is a catastrophic disease that severely endangers the patient's life and safety. It is characterized by sudden onset, rapid disease progression, and high mortality.

It should be noted that “body” and “portion” can be part of the corresponding “component”, that is, “body”, “portion” and the “other part of the component” are integrally formed and manufactured; or they can be separated as an independent component with the “other part of the component”, that is, the “body” and “portion” can be manufactured independently, and then combined with the “other part of the component” into a whole. The expressions of the above-mentioned “body” and “portion” in the present application are only one example. For the convenience of reading, it is not a limitation on the scope of protection of the present application. As long as the above features are included and the functions are the same, it should be understood as the technical solution equivalent to the present application.

It should be noted that the components included in the “units”, “components”, “mechanisms” and “devices” of the present application can also be flexibly combined, and modular production can be carried out according to actual needs to facilitate modular assembly. The division of the above-mentioned components in the present application is only one of the embodiments. For the convenience of reading, it is not a limitation of the protection scope of the present application. As long as the above-mentioned components are included and have the same function, it should be understood that it is an equivalent technical solution of the present application.

In the description of the present application, it should be understood that the terms “center”, “longitudinal”, “transverse”, “length”, “width”, “thickness”, “upper”, “lower”, “front”, “back”, “left”, “right”, “vertical”, “horizontal”, “top”, “bottom”, “inner”, “outer”, “clockwise”, “counterclockwise”, “axial”, “radial”, “circumferential”, etc. indicate the orientation or positional relationship based on the orientation or positional relationship shown in the drawings, and are only for the convenience of describing the present application and simplifying the description, rather than indicating or implying the pointed device or the element must have a specific orientation, be constructed and operated in a specific orientation, and therefore cannot be understood as a limitation of the present application.

In addition, the terms “first” and “second” are only used for descriptive purposes, and cannot be understood as indicating or implying relative importance or implicitly indicating the number of indicated technical features. Therefore, the features defined with “first” and “second” may explicitly or implicitly include at least one of the features. In the description of the present application, “plurality” means at least two, such as two, three, etc., unless otherwise specifically defined.

In the present application, unless otherwise clearly specified and limited, the terms “installed”, “connected”, “connecting”, “fixed” and other terms should be understood in a broad sense, for example, it can be a fixed connection or a detachable connection, or integrated; it can be mechanically connected or electrically connected; it can be directly connected or indirectly connected through an intermediary, it can be the internal communication of two components or the interaction relationship between two components, unless otherwise specified limitation. For those skilled in the art, the specific meaning of the above-mentioned terms in the present application can be understood according to specific circumstances.

In the present application, unless expressly stipulated and defined otherwise, the first feature “on” or “under” the second feature may be in direct contact with the first and second features, or the first and second features may be indirectly contacted through an intermediary. Moreover, the “above” or “on” of the first feature on the second feature may mean that the first feature is directly above or obliquely above the second feature, or it simply means that the level of the first feature is higher than the second feature. The first feature “below” or “under” the second feature can mean that the first feature is directly below or obliquely below the second feature, or it simply means that the level of the first feature is smaller than the second feature.

It should be noted that when an element is referred to as being “fixed to”, “arranged on”, “disposed on” or “installed on” another element, it can be directly on the other element or there may also be a centered element. When an element is considered to be “connected” to another element, it can be directly connected to the other element or an intermediate element may be present at the same time. Further, when one element is regarded as a “fixed transmission connection” and another element, the two can be fixed in a detachable connection or non-detachable connection, which can realize power transmission, such as sleeved connection and engagement, one-piece molding fixing, welding, etc., which can be realized in the prior art, and it is no longer burdensome here. When a component and another component are perpendicular or approximately perpendicular to each other, it means that the ideal state of the two is perpendicular, but due to the influence of manufacturing and assembly, there may be a certain vertical error. The terms “vertical”, “horizontal”, “left”, “right” and similar expressions used herein are for illustrative purposes only, and do not mean that they are the only embodiments.

The technical features of the above embodiments can be combined arbitrarily. In order to make the description concise, all possible combinations of the technical features in the above embodiments are not described. However, as long as there is no contradiction in the combination of these technical features, which should be considered within the scope described in this specification.

The above examples only express a few implementation modes of the present application, and the description is relatively specific and detailed, but it should not be understood as a limitation on the scope of the application patent. It should be pointed out that for those of ordinary skill in the art, without departing from the concept of the present application, several modifications and improvements can be made, and these all fall within the protection scope of the present application. Therefore, the protection scope of the patent of the present application should be subject to the appended claims.

1. A control device, comprising:
a fixing unit, comprising a screw rod provided with an accommodating hole and a sliding groove communicating with the accommodating hole, wherein a depth of the accommodating hole and a length of the sliding groove are arranged along an axis direction of the screw rod; and
a movable unit, comprising a connecting member, a first handle, a mating member, and a control member; wherein the connecting member is provided with a first connecting body and a second connecting body fixed to the first connecting body, the first connecting body is arranged in the accommodating hole, and the first connecting body is slidingly engaged with the screw rod, the second connecting body is rotatably connected to the first handle passing through the sliding groove, the first handle is hollow and is sleeved on an outside of the screw rod, and movable relative to the screw rod, the mating member is arranged in the first handle, and the mating member is provided with an internal thread structure engaged with the screw rod, the control member is movably arranged on the first handle, and the first handle drives the mating member to rotate through the control member;
wherein when the control member is in a first position, the internal thread structure is separated from the screw rod; and
when the control member is in a second position, the internal thread structure is engaged with the screw rod through a thread transmission.

2. The control device according to claim 1, wherein the movable unit further comprises a sleeve, the sleeve is sleeved on the outside of the screw rod, and the sleeve is provided with a through hole, the mating member is rotatably arranged on the sleeve, and the internal thread structure is capable of passing through the through hole to be engaged with the screw rod, the mating member is further provided with a protrusion spaced apart from the internal thread structure in a circumferential direction, the protrusion is arranged in a direction away from the screw rod, and the control member is provided with a pressing body and an extruding body spaced apart from the pressing body;
when the control member is in the first position, the extruding body is offset from the mating member, and the pressing body is press-fitted with the protrusion, such that the internal thread structure is separated from the screw rod; and
when the control member is in the second position, the pressing body is offset from the protrusion, and the extruding body is press-fitted with the mating member, such that the internal thread structure is engaged with the screw rod through a thread transmission, and the first handle is capable of driving the control member and the mating member to rotate.

3. The control device according to claim 2, wherein the control member is arranged in the sleeve and capable of being elastically reset, the control member is capable of being automatically reset to the second position under an elastic force; and an inclined surface introduction structure is provided between the extruding body and the mating member.

4. The control device according to claim 1, wherein the mating member is arranged on the screw rod and capable of being elastically reset, the control member is provided with an insertion portion;
when the control member is in the first position, the insertion portion is inserted between the internal thread structure and the screw rod, such that the internal thread structure is separated from the screw rod; and
when the control member is in the second position, the insertion portion is offset from both the internal thread structure and the screw rod, and the mating member is reset under an elastic force, such that the internal thread structure is engaged with the screw rod through a thread transmission.

5. The control device according to claim 1, wherein the fixing unit further comprises a second handle, the second handle is fixed on the screw rod, and the first handle is movable relative to the second handle.

6. The control device according to claim 1, wherein the control device further comprises a bending unit, the bending unit comprises a driving member and a flexible traction member, the driving member is movably disposed on the first handle, and the driving member is movable with the first handle, and one end of the flexible traction member is fixed on the driving member.

7. The control device according to claim 6, wherein the driving member is rotatable relative to the first handle, and the driving member is provided with a connecting sleeve sleeved on the screw rod and a force applying portion fixed on an outside of the connecting sleeve; one end of the flexible traction member is fixed on the connecting sleeve; or the connecting sleeve is provided with an external thread structure, and the bending unit further comprises a third handle fixedly connected with the first handle and a threaded sleeve that is screwed and engaged with the external thread structure, the third handle is hollow, the threaded sleeve is arranged in the third handle and is slidably connected to the third handle, one end of the flexible traction member is fixed on the threaded sleeve.

8. The control device according to claim 1, wherein the fixing unit further comprises a guide rod, the guide rod is fixed in the screw rod, and the first connecting body is provided with a mating hole that is slidingly fitted with the guide rod.

9. The control device according to claim 8, wherein the guide rod is provided with a flow channel, and the fixing unit further comprises a guide tube in communicated with the flow channel, and one end of the guide tube is fixed to the screw rod and is communicated with outside.

10. A delivery apparatus, comprising a control device, wherein the first connecting body is provided with a connection portion, and the delivery apparatus further comprises a delivery tube for delivering a vascular stent, and one end of the delivery tube is fixedly connected with a connecting member through the connection portion; and
the control device comprises:
a fixing unit, comprising a screw rod provided with an accommodating hole and a sliding groove communicating with the accommodating hole, wherein a depth of the accommodating hole and a length of the sliding groove are arranged along an axis direction of the screw rod; and
a movable unit, comprising a connecting member, a first handle, a mating member, and a control member; wherein the connecting member is provided with a first connecting body and a second connecting body fixed to the first connecting body, the first connecting body is arranged in the accommodating hole, and the first connecting body is slidingly engaged with the screw rod, the second connecting body is rotatably connected to the first handle passing through the sliding groove, the first handle is hollow and is sleeved on an outside of the screw rod, and movable relative to the screw rod, the mating member is arranged in the first handle, and the mating member is provided with an internal thread structure engaged with the screw rod, the control member is movably arranged on the first handle, and the first handle drives the mating member to rotate through the control member;
wherein when the control member is in a first position, the internal thread structure is separated from the screw rod; and
when the control member is in a second position, the internal thread structure is engaged with the screw rod through a thread transmission.

11. The control device according to claim 2, wherein the fixing unit further comprises a guide rod, the guide rod is fixed in the screw rod, and the first connecting body is provided with a mating hole that is slidingly fitted with the guide rod.

12. The control device according to claim 3, wherein the fixing unit further comprises a guide rod, the guide rod is fixed in the screw rod, and the first connecting body is provided with a mating hole that is slidingly fitted with the guide rod.

13. The control device according to claim 4, wherein the fixing unit further comprises a guide rod, the guide rod is fixed in the screw rod, and the first connecting body is provided with a mating hole that is slidingly fitted with the guide rod.

14. The control device according to claim 5, wherein the fixing unit further comprises a guide rod, the guide rod is fixed in the screw rod, and the first connecting body is provided with a mating hole that is slidingly fitted with the guide rod.

15. The control device according to claim 6, wherein the fixing unit further comprises a guide rod, the guide rod is fixed in the screw rod, and the first connecting body is provided with a mating hole that is slidingly fitted with the guide rod.

16. The control device according to claim 7, wherein the fixing unit further comprises a guide rod, the guide rod is fixed in the screw rod, and the first connecting body is provided with a mating hole that is slidingly fitted with the guide rod.

17. The control device according to claim 11, wherein the guide rod is provided with a flow channel, and the fixing unit further comprises a guide tube in communicated with the flow channel, and one end of the guide tube is fixed to the screw rod and is communicated with outside.

18. The control device according to claim 12, wherein the guide rod is provided with a flow channel, and the fixing unit further comprises a guide tube in communicated with the flow channel, and one end of the guide tube is fixed to the screw rod and is communicated with outside.

19. The control device according to claim 13, wherein the guide rod is provided with a flow channel, and the fixing unit further comprises a guide tube in communicated with the flow channel, and one end of the guide tube is fixed to the screw rod and is communicated with outside.

20. The control device according to claim 14, wherein the guide rod is provided with a flow channel, and the fixing unit further comprises a guide tube in communicated with the flow channel, and one end of the guide tube is fixed to the screw rod and is communicated with outside.
